(19) United States
(12) Patent Application Publication
(10) Pub. No.: US 2025/0213389 A1
(43) Pub. Date: Jul. 3, 2025

(54) OCULAR DRUG DELIVERY DEVICES SECURELY IMPLANTABLE INTO ANTERIOR SEGMENT OF EYE

(71) Applicant: OnPoint Vision, Inc., Aliso Viejo, CA (US)

(72) Inventor: Kevin J. Cady, Franklin, TN (US)

(21) Appl. No.: 19/005,408

(22) Filed: Dec. 30, 2024

Related U.S. Application Data
(60) Provisional application No. 63/616,939, filed on Jan. 2, 2024.

(51) Int. Cl. A61F 9/00; A61F 2/16

(52) U.S. Cl. CPC A61F 9/0017; A61F 2/16; A61F 2250/0068

(57) ABSTRACT

An apparatus includes an ocular drug delivery device having a reservoir configured to hold at least one medication for an eye and a micro-insert configured to be inserted under an anterior leaflet of a capsular wall in the eye. An anterior surface of the micro-insert is configured to contact an inner capsular wall surface of the anterior leaflet. A posterior surface of the micro-insert includes a ridge configured to contact an edge of an artificial intraocular lens.

### CROSS-REFERENCE TO RELATED APPLICATION AND PRIORITY CLAIM

This application claims priority under 35 U.S.C. § 119 (e) to U.S. Provisional Patent Application No. 63/616,939 filed on Jan. 2, 2024, which is hereby incorporated by reference in its entirety.

### TECHNICAL FIELD

This disclosure relates generally to implantable ocular devices. More specifically, this disclosure relates to ocular drug delivery devices that are securely implantable into the anterior segment of an eye.

### BACKGROUND

Various medical conditions may require that medication be delivered to patients' eyes. In some cases, this may require injections of medications into the patients' eyes, and these injections are referred to as “intravitreal” injections. Intravitreal injections may be used in the treatment of a number of conditions, such as age-related macular degeneration (AMD), retinal vein occlusion, chronic uveitis, glaucoma, ocular hypertension, or diabetic eye disease. Many patients tend to be anxious about receiving intravitreal injections in their eyes. Moreover, intravitreal injections can have various potential side effects, such as irritation, subconjunctival hemorrhages, or infections. In addition, some conditions might require the constant use and administration of topical eye drops. However, potential inconsistent amounts of drug delivery, the natural biphasic property of an eye's surface that acts as a barrier to drug delivery, and the reliance on patient compliance remain major concerns with medical practitioners. Patients also often complain about the inconvenience of being held responsible for self-administration of medication to treat their eye conditions.

### SUMMARY

This disclosure relates to ocular drug delivery devices that are securely implantable into the anterior segment of an eye.

In a first embodiment, an apparatus includes an ocular drug delivery device having a reservoir configured to hold at least one medication for an eye and a micro-insert configured to be inserted under an anterior leaflet of a capsular wall in the eye. An anterior surface of the micro-insert is configured to contact an inner capsular wall surface of the anterior leaflet. A posterior surface of the micro-insert includes a ridge configured to contact an edge of an artificial intraocular lens.

In a second embodiment, a system includes an artificial intraocular lens configured to be implanted within a capsular bag in an eye. The system also includes an ocular drug delivery device having a reservoir configured to hold at least one medication for the eye and a micro-insert configured to be inserted under an anterior leaflet of a capsular wall in the eye. An anterior surface of the micro-insert is configured to contact an inner capsular wall surface of the anterior leaflet. A posterior surface of the micro-insert includes a ridge configured to contact an edge of the artificial intraocular lens.

In a third embodiment, a method includes forming an incision in an eye and inserting an ocular drug delivery device in the eye. The ocular drug delivery device includes a reservoir configured to hold at least one medication for the eye and a micro-insert configured to be inserted under an anterior leaflet of a capsular wall in the eye. An anterior surface of the micro-insert is configured to contact an inner capsular wall surface of the anterior leaflet. A posterior surface of the micro-insert includes a ridge configured to contact an edge of an artificial intraocular lens. In some cases, the method may also include forming a pocket or tunnel under the anterior leaflet in the eye and positioning at least part of the ocular drug delivery device within the pocket or tunnel.

Any single one or any suitable combination of the following features may be used with the first, second, or third embodiment. At least part of the micro-insert may extend laterally away from the reservoir. The reservoir may include an interior space that extends completely through the reservoir such that the reservoir has multiple openings providing access to the interior space, where the interior space is configured to receive the at least one medication. The reservoir may include an interior space that extends partially through the reservoir such that the reservoir has one opening providing access to the interior space, where the interior space is configured to receive the at least one medication. The reservoir may include an interior space configured to receive the at least one medication, where the interior space extends horizontally or vertically through the reservoir. The micro-insert may include a recess in the anterior surface of the micro-insert, where the recess extends away from the reservoir. At least one side of the reservoir may include a convex surface and smaller concave surfaces. The reservoir may be positioned over at least part of the micro-insert. The reservoir may be integral with the micro-insert. The reservoir may include at least one opening providing access to an interior space of the reservoir, where the interior space is configured to receive the at least one medication. The reservoir may include at least one membrane configured to control a flow of the at least one medication through the at least one opening. The reservoir may include a stent configured to release the at least one medication.

Other technical features may be readily apparent to one skilled in the art from the following figures, descriptions, and claims.

### BRIEF DESCRIPTION OF THE DRAWINGS

For a more complete understanding of this disclosure and its features, reference is now made to the following description, taken in conjunction with the accompanying drawings, in which:

FIGS. 1A through 1D illustrate a first example ocular drug delivery device that is securely implantable into the anterior segment of an eye according to this disclosure;

FIGS. 2A through 2E illustrate a second example ocular drug delivery device that is securely implantable into the anterior segment of an eye according to this disclosure;

FIGS. 3A through 3E illustrate a third example ocular drug delivery device that is securely implantable into the anterior segment of an eye according to this disclosure;

FIGS. 4A through 4D illustrate a fourth example ocular drug delivery device that is securely implantable into the anterior segment of an eye according to this disclosure;

FIGS. 5A through 5D illustrate a fifth example ocular drug delivery device that is securely implantable into the anterior segment of an eye according to this disclosure;

FIGS. 6A through 6D illustrate a sixth example ocular drug delivery device that is securely implantable into the anterior segment of an eye according to this disclosure;

FIGS. 7A through 7D illustrate a seventh example ocular drug delivery device that is securely implantable into the anterior segment of an eye according to this disclosure;

FIGS. 8A through 8D illustrate an eighth example ocular drug delivery device that is securely implantable into the anterior segment of an eye according to this disclosure;

FIGS. 9A through 9D illustrate a ninth example ocular drug delivery device that is securely implantable into the anterior segment of an eye according to this disclosure;

FIGS. 10A through 10D illustrate a tenth example ocular drug delivery device that is securely implantable into the anterior segment of an eye according to this disclosure;

FIGS. 11A through 11D illustrate an eleventh example ocular drug delivery device that is securely implantable into the anterior segment of an eye according to this disclosure;

FIG. 12 illustrates an example membrane used with ocular drug delivery devices that are securely implantable into the anterior segment of an eye according to this disclosure;

FIG. 13 illustrates an example stent used with an ocular drug delivery device that is securely implantable into the anterior segment of an eye according to this disclosure;

FIG. 14 illustrates an example secure implantation of an ocular drug delivery device into the anterior segment of an eye according to this disclosure; and

FIG. 15 illustrates an example method for using an ocular drug delivery device that is securely implantable into the anterior segment of an eye according to this disclosure.

### DETAILED DESCRIPTION

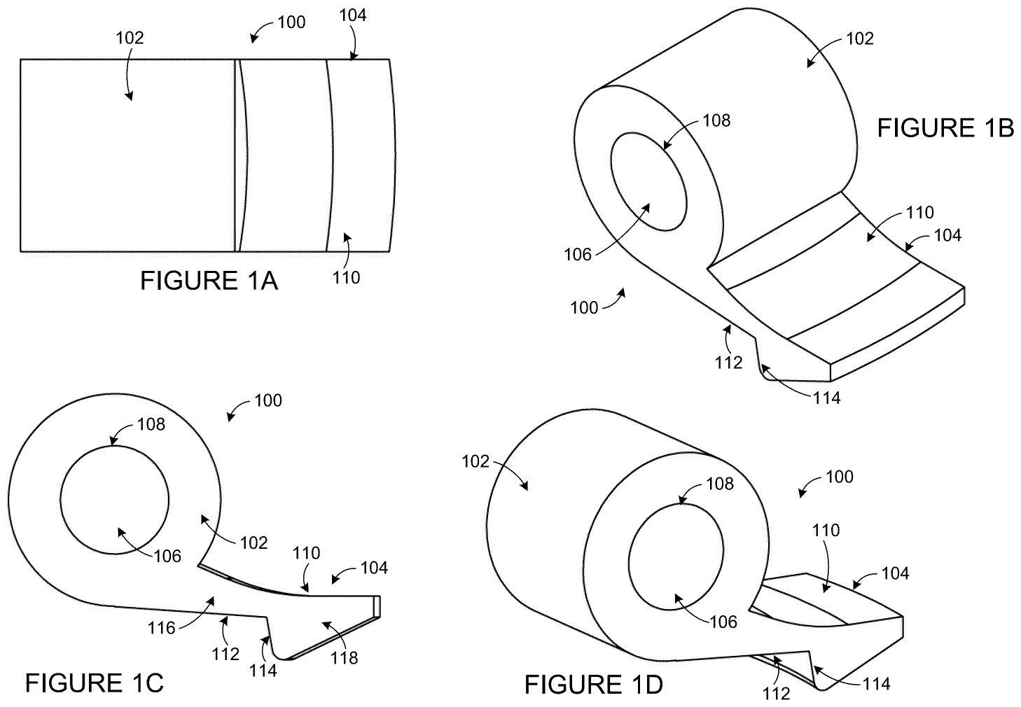

FIGS. 1A through 15, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

As noted above, various medical conditions may require that medication be delivered to patients' eyes. In some cases, this may require injections of medications into the patients' eyes, and these injections are referred to as “intravitreal” injections. Intravitreal injections may be used in the treatment of a number of conditions, such as age-related macular degeneration (AMD), retinal vein occlusion, chronic uveitis, glaucoma, ocular hypertension, or diabetic eye disease. Many patients tend to be anxious about receiving intravitreal injections in their eyes. Moreover, intravitreal injections can have various potential side effects, such as irritation, subconjunctival hemorrhages, or infections. In addition, some conditions might require the constant use and administration of topical eye drops. However, potential inconsistent amounts of drug delivery, the natural biphasic property of an eye's surface that acts as a barrier to drug delivery, and the reliance on patient compliance remain major concerns with medical practitioners. Patients also often complain about the inconvenience of being held responsible for self-administration of medication to treat their eye conditions.

This disclosure provides various ocular drug delivery devices that are securely implantable into the anterior segment of an eye. As described in more detail below, each of the ocular drug delivery devices may be used in conjunction with an intraocular lens (IOL). Each of the ocular drug delivery devices includes a reservoir configured to hold at least one medication and to release the at least one medication into a patient's eye. In some cases, the reservoir may include one or more openings through which at least one medication can be released into the patient's eye. Also, in some cases, the reservoir may include one or more membranes configured to control the release of at least one medication through the one or more openings. Further, in some cases, the reservoir may include a stent configured to release at least one medication.

Each of the ocular drug delivery devices also includes at least one micro-insert configured to be positioned under the anterior leaflet of a capsular wall in the patient's eye. In some cases, the micro-insert may extend laterally away from the reservoir. In other cases, the reservoir may be positioned over and attached to or integrated with the micro-insert. The reservoir or the micro-insert can include an anterior surface that contacts the anterior leaflet of the capsular wall in the patient's eye. In some cases, the anterior surface may be textured or include a serrated surface such as to allow the ocular drug delivery device to be captured and confined by the anterior leaflet of the capsular wall. In particular cases, the ocular drug delivery device can actually attach to the anterior leaflet of the capsular wall, such as through fibrosis or re-fibrosis during the healing process, to help secure the ocular drug delivery device in place. The micro-insert can include a posterior surface having a ridge, which can be positioned under the anterior leaflet of the capsular wall and against an edge of an underlying intraocular lens. The ridge, along with surface tension of the anterior leaflet resting on the anterior surface of the reservoir or micro-insert, can help to hold the ocular drug delivery device in place.

In this way, each ocular drug delivery device can be securely implanted within a patient's eye. Once implanted, the reservoir of the ocular drug delivery device can release at least one medication into the patient's eye, such as when the at least one medication is delivered slowly over time. As a result, at least one medication can be used to treat one or more medical conditions associated with the patient's eye, such as AMD, retinal vein occlusion, diabetic eye disease, chronic inflammation of internal ocular tissue, glaucoma, various retinal diseases, or other conditions. Moreover, because each ocular drug delivery device can be secured under the anterior leaflet of the capsular wall in front of an intraocular lens, the ocular drug delivery device can be implanted within the anterior segment of the patient's eye. This can simplify implantation of the ocular drug delivery device and reduce or avoid the need to perform a more-risky implantation in the posterior segment of the patient's eye. This can also reduce or avoid the use of therapeutic stents, which can interfere with the natural drainage of aqueous from the eye. In addition, this can help to maintain the ocular drug delivery device in a desired position that does not interfere with the patient's vision, compared to some approaches that implant free-floating devices within patients' eyes. Finally, this can help to provide more consistent amounts of drug delivery, reduce or eliminate barriers to drug delivery, and stop reliance on patient compliance.

FIGS. 1A through 1D illustrate a first example ocular drug delivery device 100 that is securely implantable into the anterior segment of an eye according to this disclosure. As shown in FIGS. 1A through 1D, the ocular drug delivery device 100 includes a reservoir 102 and a micro-insert 104. The reservoir 102 is generally configured to receive at least one medication and to release or allow release of the at least one medication into a patient's eye following implantation of the ocular drug delivery device 100. In this particular example, the reservoir 102 represents a structure that is generally cylindrical and that has an interior space 106, where one or more openings 108 of the reservoir 102 provide access to the interior space 106. The at least one medication can be positioned within the interior space 106 of the reservoir 102, such as prior to, during, or after implantation. Once within a patient's eye, the at least one medication can be released from the reservoir 102, such as over any desired period of time (and possibly over an extended period of time). Note that while it may be assumed in FIGS. 1A through 1D that the interior space 106 extends completely through the reservoir 102 between two openings 108, the interior space 106 may represent a recess in the reservoir 102 that is accessible through a single opening 108.

The micro-insert 104 is sized and shaped to extend from the reservoir 102 and fit under the anterior leaflet of the capsular wall in a patient's eye. The anterior leaflet of the capsular wall in a patient's eye is typically created during a capsulotomy in which the natural crystalline lens in the patient's eye is removed and replaced with an intraocular lens. The anterior leaflet represents the outer portion of the front side of the capsular bag that remains after an opening (referred to as a capsulorhexis) is formed in the capsular bag so that the natural crystalline lens can be removed. In some cases, this could occur long before the ocular drug delivery device 100 is to be implanted. After the capsulotomy, the anterior leaflet of the capsular wall typically shrinks and undergoes fibrosis during the post-operative healing process.

When the ocular drug delivery device 100 is inserted into a patient's eye, the ocular drug delivery device 100 can be positioned so that the micro-insert 104 extends under the anterior leaflet in the patient's eye. The micro-insert 104 could also be physically attached to the anterior leaflet over time, such as by way of a “re-fibrosis” of the anterior leaflet. This re-fibrosis of tissue will bond to and cover part or all of the micro-insert 104, further securing the ocular drug delivery device 100 in place. Note, however, that the ocular drug delivery device 100 could also be implanted during the same procedure in which the intraocular lens is being implanted. In that case, the ocular drug delivery device 100 could be secured by the micro-insert 104 and possibly during fibrosis (and not re-fibrosis) within the patient's eye. Also note that the term “micro-insert” here does not impart any specific dimensional requirements on the micro-insert 104 and instead merely indicates that the micro-insert 104 is generally small enough to fit under the anterior leaflet of the capsular wall in a patient's eye.

An anterior surface 110 of the micro-insert 104 may contact an inner capsular wall surface of the anterior leaflet of the capsular wall in a patient's eye and be held in place, such as via surface tension. In some cases, the anterior surface 110 of the micro-insert 104 may represent a textured or serrated surface, such as a surface having holes formed partially or completely through the micro-insert 104 or other non-smooth surface features. The textured or serrated anterior surface 110 may facilitate bonding of the micro-insert 104 to the anterior leaflet of the capsular wall, such as through fibrosis or re-fibrosis during the healing process.

The micro-insert 104 also helps to secure the ocular drug delivery device 100 in place via interaction with an intraocular lens within the patient's eye. For example, a posterior surface 112 of the micro-insert 104 includes a ridge 114. When the ocular drug delivery device 100 is implanted in a patient's eye, the micro-insert 104 can be inserted under the anterior leaflet of the capsular wall, which allows the ridge 114 on the leading edge of the posterior side of the micro-insert 104 to be positioned adjacent to or against an edge of an intraocular lens within the patient's eye (such as an outer edge of a lens of the intraocular lens). This can help to secure the ocular drug delivery device 100 in place and can help to prevent the ocular drug delivery device 100 from slipping out from under the anterior leaflet of the capsular wall. In some cases, both the pressure from the anterior leaflet of the capsular wall against the anterior surface 110 of the micro-insert 104 and contact of the ridge 114 against the edge of the underlying intraocular lens can be used to hold the ocular drug delivery device 100 securely in place. Among other things, this can help to keep the ocular drug delivery device 100 in place during the healing process, during which the ocular drug delivery device 100 may be further secured via fibrosis or re-fibrosis to the anterior leaflet of the capsular wall. Note, however, that this is not required, and the ocular drug delivery device 100 may be held in place through contact of the micro-insert 104 with the anterior leaflet of the capsular wall and/or with the underlying intraocular lens.

As shown in FIGS. 1B through 1D, the micro-insert 104 in this example is formed by an inner portion 116 and an outer portion 118 of the micro-insert 104. The inner portion 116 may extend down and laterally away from the reservoir 102. The inner portion 116 may have a larger thickness closer to the reservoir 102 and may taper in thickness moving away from the reservoir 102. However, these features can vary as needed or desired. As an example, the inner portion 116 may extend laterally away from the reservoir 102 without extending down from the reservoir 102. As another example, the inner portion 116 may have a substantially or completely uniform thickness. The outer portion 118 may have a larger thickness closer to the inner portion 116 and may taper in thickness moving towards the end of the micro-insert 104. This tapering may facilitate insertion of the micro-insert 104 under the anterior leaflet of the capsular wall. Again, however, these features can vary as needed or desired. In some embodiments, the ridge 114 may be formed or defined where the inner portion 116 (having a smaller thickness) meets the outer portion 118 (having a larger thickness). Although not shown here, the ridge 114 may include a small lip, such as at or near the bottom of the ridge 114, which could project from the ridge 114 back towards the reservoir 102. Such a lip may help to facilitate more effective contact with the edge of the underlying intraocular lens or reduce slippage of the micro-insert 104 along the edge of the underlying intraocular lens. The micro-insert 104 may also be flexible, such as when pressure from the anterior leaflet of the capsular wall against the anterior surface 110 of the micro-insert 104 pushes the ridge 114 and/or the lip into or below the edge of the underlying intraocular lens.

In the example shown in FIGS. 1A through 1D, the interior space 106 of the reservoir 102 may extend partially or completely through the reservoir 102 in a direction that is substantially or completely perpendicular to the direction in which the micro-insert 104 extends away from the reservoir 102. However, this need not be the case.

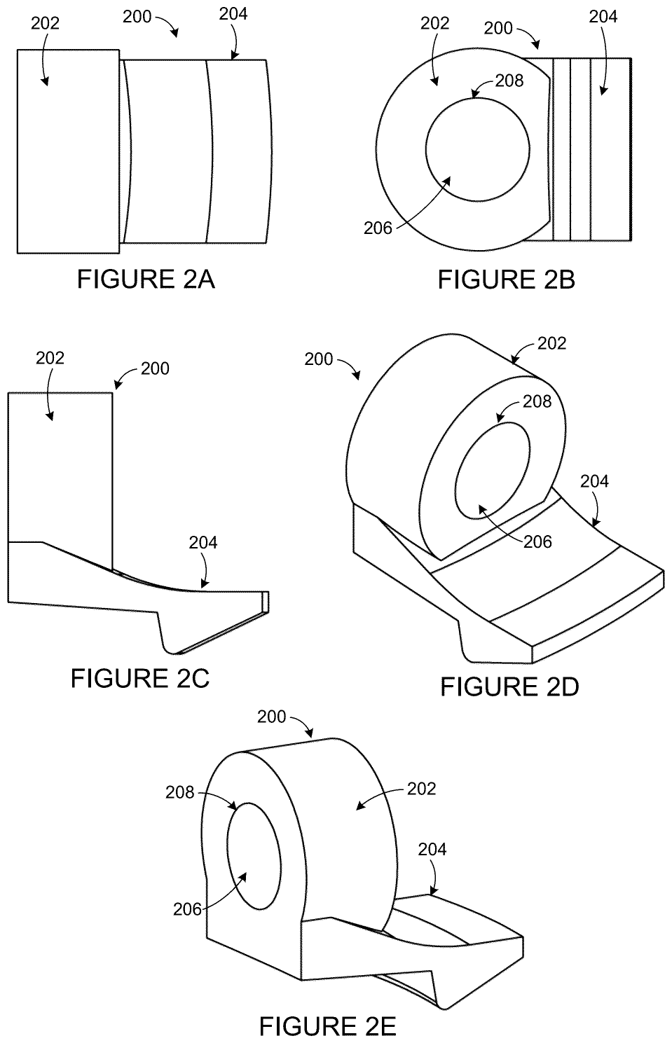

FIGS. 2A through 2E illustrate a second example ocular drug delivery device 200 that is securely implantable into the anterior segment of an eye according to this disclosure. As shown in FIGS. 2A through 2E, the ocular drug delivery device 200 includes a reservoir 202 and a micro-insert 204. The micro-insert 204 may be the same as or similar to the micro-insert 104 described above. As a result, all discussions regarding the micro-insert 104 above can apply to the micro-insert 204 and are omitted for brevity.

The reservoir 202 may be similar to the reservoir 102 discussed above. For example, the reservoir 202 can include an interior space 206, where one or more openings 208 of the reservoir 202 provide access to the interior space 206. While it is assumed here that the interior space 206 extends completely through the reservoir 202 between two openings 208, the interior space 206 may represent a recess in the reservoir 202 that is accessible through a single opening 208. In this example, the interior space 206 of the reservoir 202 can extend partially or completely through the reservoir 202 in a direction that is substantially or completely parallel to the direction in which the micro-insert 204 extends away from the reservoir 202 (and possibly along the central axis of the micro-insert 204). Note that while the reservoir 202 is shown here as being generally smaller in terms of distance between the two openings 208 compared to the reservoir 102, the reservoir 202 may have any suitable size and dimensions.

In FIGS. 1A through 2E, the sides of the reservoirs 102, 202 are shown as being generally flat, where the openings 108, 208 providing access to the interior spaces 106, 206 of the reservoirs 102, 202 are located on the generally flat sides of the reservoirs 102, 202. However, this need not be the case.

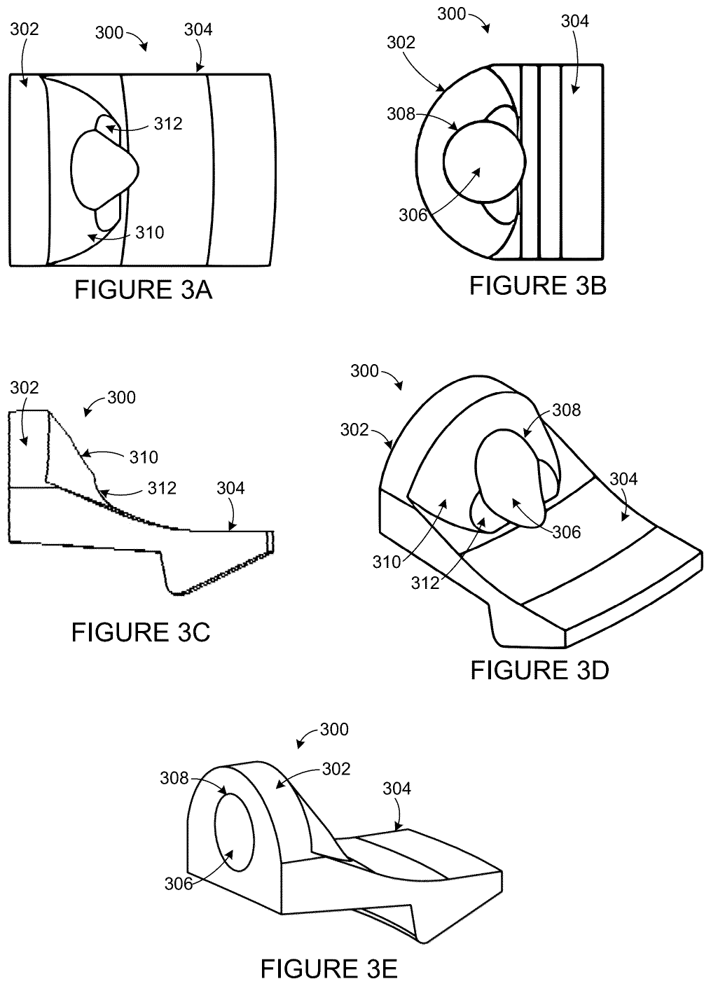

FIGS. 3A through 3E illustrate a third example ocular drug delivery device 300 that is securely implantable into the anterior segment of an eye according to this disclosure. As shown in FIGS. 3A through 3E, the ocular drug delivery device 300 includes a reservoir 302 and a micro-insert 304. The micro-insert 304 may be the same as or similar to the micro-insert 104 described above. As a result, all discussions regarding the micro-insert 104 above can apply to the micro-insert 304 and are omitted for brevity.

The reservoir 302 is similar to the reservoir 202 described above. For example, the reservoir 302 can include an interior space 306, where one or more openings 308 of the reservoir 302 provide access to the interior space 306. However, in this example, the reservoir 302 has one generally flat side. The other side of the reservoir 302 includes various curved surfaces, such as a convex surface 310 and two smaller concave surfaces 312. As a result, only one opening 308 is generally circular, while the other opening 308 has a non-circular shape due to its presence within the surfaces 310, 312. The surfaces 310, 312 may be used in various ways. For instance, in some cases, the convex surface 310 may help to maintain some separation of the reservoir 302 from the anterior leaflet of the capsular wall in a patient's eye. Also or alternatively, in some cases, the concave surfaces 312 may help to contact the anterior leaflet and resist pushing of the convex surface 310 further under the anterior leaflet.

While it is assumed here that the interior space 306 extends completely through the reservoir 302 between two openings 308, the interior space 306 may represent a recess in the reservoir 302 that is accessible through a single opening 308. In some cases, the single opening 308 may be formed within the curved surfaces 310, 312. In other cases, the single opening 308 may be formed within the flat side of the reservoir 302, and the opening 308 shown within the curved surfaces 310, 312 may instead represent a curved or flat surface that blocks the interior space 306.

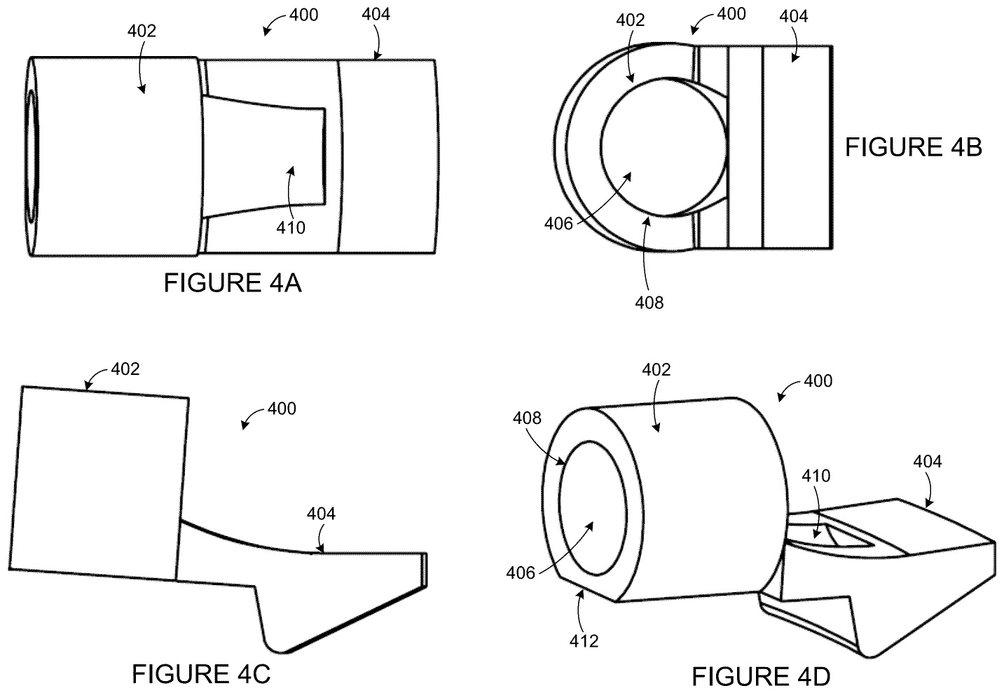

FIGS. 4A through 4D illustrate a fourth example ocular drug delivery device 400 that is securely implantable into the anterior segment of an eye according to this disclosure. As shown in FIGS. 4A through 4D, the ocular drug delivery device 400 includes a reservoir 402 and a micro-insert 404. The micro-insert 404 may be the same as or similar to the micro-insert 104 described above. As a result, all discussions regarding the micro-insert 104 above can apply to the micro-insert 404 and are omitted for brevity. The reservoir 402 may be the same as or similar to the reservoir 202 described above. For example, the reservoir 402 can include an interior space 406, where one or more openings 408 of the reservoir 402 provide access to the interior space 406. While it is assumed here that the interior space 406 extends completely through the reservoir 402 between two openings 408, the interior space 406 may represent a recess in the reservoir 402 that is accessible through a single opening 408. In this example, the reservoir 402 is longer than the reservoir 202, although the reservoir 402 may have the same or similar length as the reservoir 202 or a shorter length than the reservoir 202.

In this example, a recess 410 is formed or otherwise positioned in the anterior surface of the micro-insert 404 and generally extends from at or near the reservoir 402 to some distance away from the reservoir 402. The recess 410 may have any suitable size and shape. In some cases, the end of the reservoir 402 could allow for the placement of an attached membrane, such as one made of polyvinyl alcohol or any other suitable material, to allow for a prolonged distribution of drug therapy. Note that the length of the recess 410 along the micro-insert 404 shown here is for illustration only. The recess 410 may extend shorter or longer distances along the micro-insert 404 than shown here.

As can be seen in FIG. 4D, the reservoir 402 need not be perfectly cylindrical. In this example, for instance, a bottom surface 412 of the reservoir 402 may be flat or have a smaller radius of curvature compared to other portions of the reservoir 402. The bottom surface 412 of the reservoir 402 here may help to provide a larger contact area between the reservoir 402 and the underlying intraocular lens, which can help to stabilize the ocular drug delivery device 400 and resist slippage of the ocular drug delivery device 400. Note, however, that this is not necessarily needed, and the reservoir 402 may be generally cylindrical.

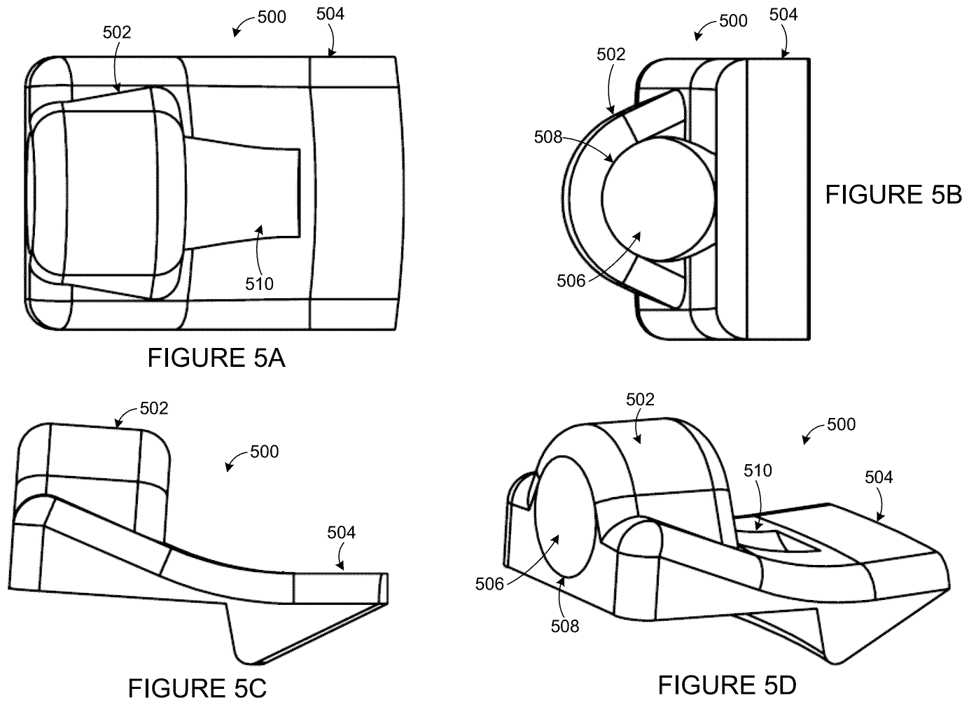

FIGS. 5A through 5D illustrate a fifth example ocular drug delivery device 500 that is securely implantable into the anterior segment of an eye according to this disclosure. The ocular drug delivery device 500 has a similar design as the ocular drug delivery device 400. For example, the ocular drug delivery device 500 includes a reservoir 502 and a micro-insert 504, where the reservoir 502 has an interior space 506 accessible through one or more openings 508. While it is assumed here that the interior space 506 extends completely through the reservoir 502 between two openings 508, the interior space 506 may represent a recess in the reservoir 502 that is accessible through a single opening 508. Also, the micro-insert 504 includes a recess 510. While the length of the recess 510 along the micro-insert 504 shown here is for illustration only, the recess 510 may extend shorter or longer distances along the micro-insert 504 than shown here. In this example, the ocular drug delivery device 500 and its individual components are more rounded compared to the ocular drug delivery device 400. Also, the reservoir 502 here is more integrated with the micro-insert 504, and the interior space 506 of the reservoir 502 might actually extend into the micro-insert 504. In addition, the reservoir 502 is not as wide as the micro-insert 504 (although this need not be the case).

Note that the relative sizes, shapes, and dimensions of the ocular drug delivery devices and their individual components described above are for illustration and explanation only. Other embodiments of the ocular drug delivery devices can vary based on a number of factors and in a number of ways.

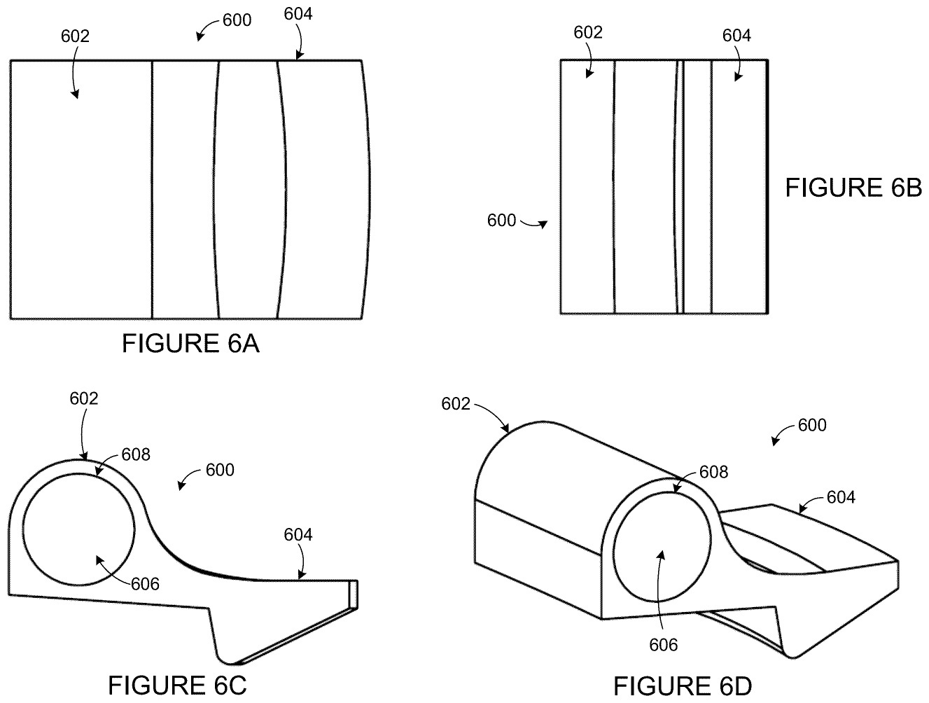

FIGS. 6A through 6D illustrate a sixth example ocular drug delivery device 600 that is securely implantable into the anterior segment of an eye according to this disclosure. As shown in FIGS. 6A through 6D, the ocular drug delivery device 600 includes a reservoir 602 and a micro-insert 604. The micro-insert 604 may be the same as or similar to the micro-insert 104 described above. As a result, all discussions regarding the micro-insert 104 above can apply to the micro-insert 604 and are omitted for brevity.

The reservoir 602 may be similar to the reservoir 102 described above. However, an interior space 606 of the reservoir 602 may be larger (compared to the reservoir 102), and one or more openings 608 providing access to the interior space 606 may be larger (compared to the one or more openings 108). Among other things, this may allow a larger quantity of one or more medications to be stored in the reservoir 602 and/or an additional extended release of the one or more medications into the patient's eye. While it is assumed here that the interior space 606 extends completely through the reservoir 602 between two openings 608, the interior space 606 may represent a recess in the reservoir 602 that is accessible through a single opening 608.

In FIGS. 1A through 6D, the reservoirs 102-602 are designed so that their interior spaces 106-606 extend horizontally, where at least one medication can be delivered through one or more openings 108-608 that are positioned along one or more sides of the ocular drug delivery devices 100-600. However, this need not be the case.

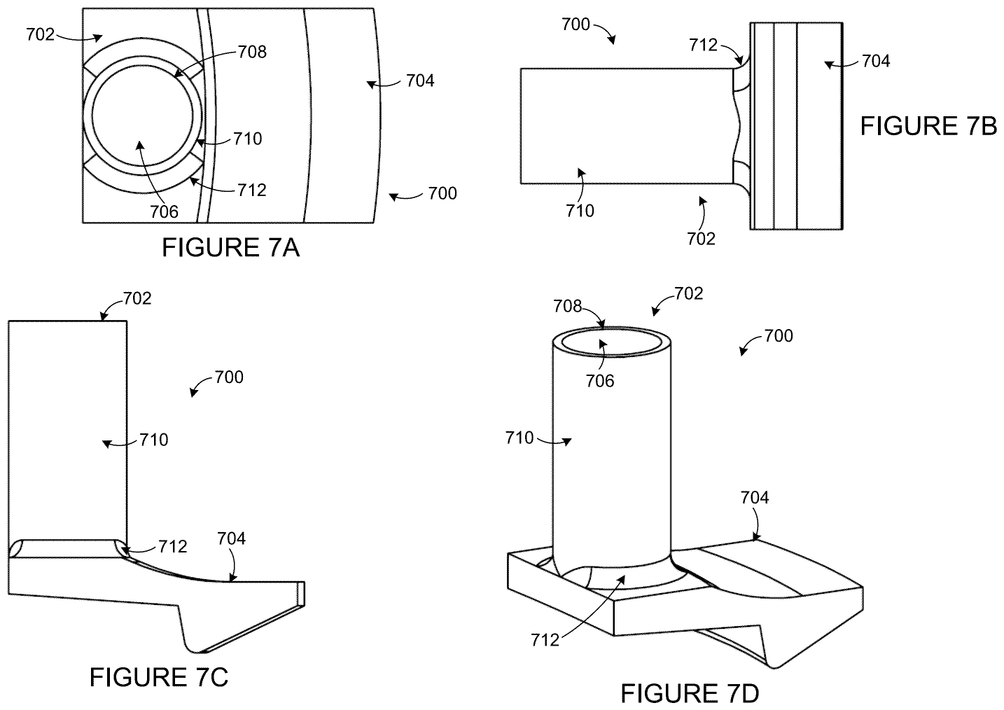

FIGS. 7A through 7D illustrate a seventh example ocular drug delivery device 700 that is securely implantable into the anterior segment of an eye according to this disclosure. As shown in FIGS. 7A through 7D, the ocular drug delivery device 700 includes a reservoir 702 and a micro-insert 704. The micro-insert 704 may be the same as or similar to the micro-insert 104 described above. As a result, all discussions regarding the micro-insert 104 above can apply to the micro-insert 704 and are omitted for brevity.

The reservoir 702 in this example includes a projection 710 that extends vertically from a base 712 of the reservoir 702. The projection 710 includes an interior space 706, where one or more openings 708 of the reservoir 702 provide access to the interior space 706. Depending on the implementation, the interior space 706 may extend completely through the reservoir 702 between two openings 708 on top and bottom of the ocular drug delivery device 700, or the interior space 706 may represent a recess in the reservoir 702 that is accessible through a single opening 708. It is also or alternatively possible for the reservoir 702 to include one or more openings on one or sides of the projection 710.

The base 712 of the reservoir 702 can be connected to or be integral with a portion of the micro-insert 704 extending under the reservoir 702. The base 712 of the reservoir 702 can also be connected to or be integral with the projection 710. In this example, the micro-insert 704 may itself include an opening and passage through the micro-insert 704 to the interior space 706 of the reservoir 702, thereby allowing at least one medication in the reservoir 702 to be released through the micro-insert 704 as well as (or instead of) through the opening 708 in the top of the projection 710.

Note that while squared corners are shown in FIGS. 7A through 7D at various locations, these corners may also be rounded. For example, the upper left and lower left corners in FIG. 7A could be rounded. As a particular example, the curvatures of these corners could match or closely match the curvature of the base 712 near those corners. However, this is not necessarily required, and these corners and the base 712 may have different amounts of curvature.

In FIGS. 1A through 7D, most or all of the reservoirs 102-702 are shown as being offset laterally from large portions of the micro-inserts 104-704. In other words, the micro-inserts 104-704 can extend relatively large distances from the reservoirs 102-702 laterally. However, this need not be the case.

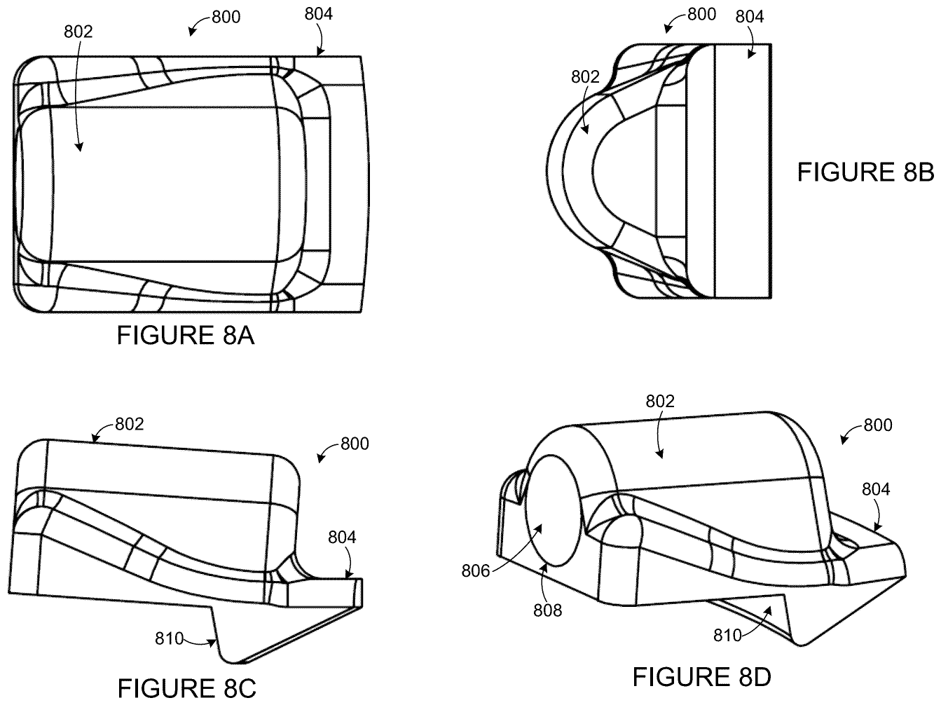

FIGS. 8A through 8D illustrate an eighth example ocular drug delivery device 800 that is securely implantable into the anterior segment of an eye according to this disclosure. As shown in FIGS. 8A through 8D, the ocular drug delivery device 800 includes various components that appear similar to various components of other ocular drug delivery devices described above, such as various components shown in FIGS. 5A through 5D. For example, the ocular drug delivery device 800 includes a reservoir 802 and a micro-insert 804, where the reservoir 802 has an interior space 806 accessible through an opening 808. However, the ocular drug delivery device 800 integrates the reservoir 802 and the micro-insert 804 such that most or all of the reservoir 802 is positioned over the micro-insert 804 (rather than laterally with respect to the micro-insert 804). Moreover, in this example, only one opening 808 is present, and the interior space 806 is blocked at the other end of the reservoir 802 (although this need not be the case). The micro-insert 804 still includes a ridge 810, which may include an optional lip as described above.

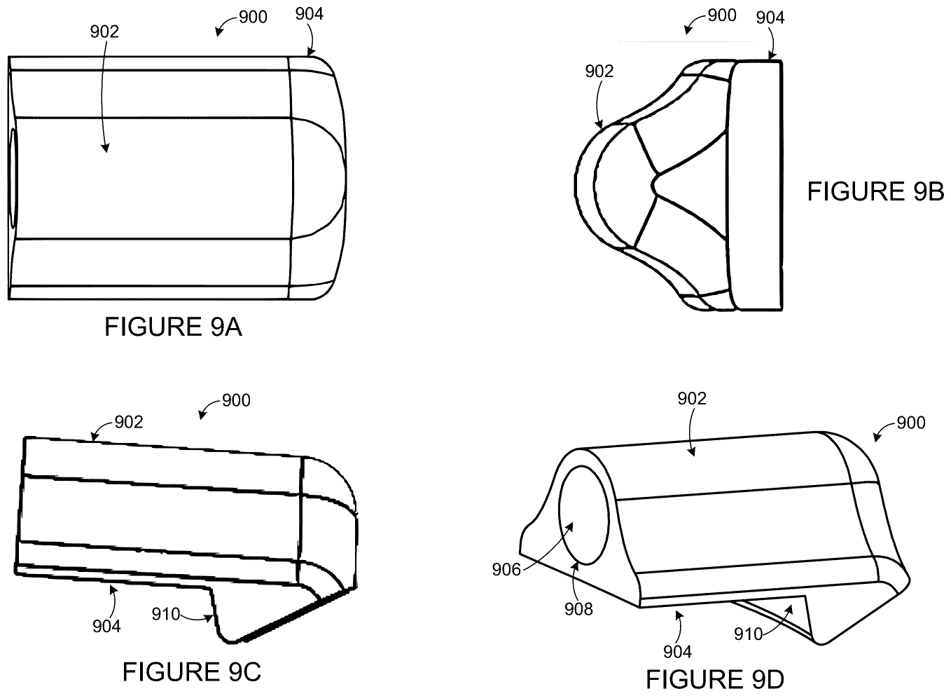

FIGS. 9A through 9D illustrate a ninth example ocular drug delivery device 900 that is securely implantable into the anterior segment of an eye according to this disclosure. As shown in FIGS. 9A through 9D, the ocular drug delivery device 900 is similar to the ocular drug delivery device 800 of FIGS. 8A through 8D. For example, the ocular drug delivery device 900 includes a reservoir 902 and a micro-insert 904, where the reservoir 902 has an interior space 906 accessible through an opening 908. Most or all of the reservoir 902 is positioned over the micro-insert 904, the interior space 906 is blocked at the other end (although this need not be the case), and the micro-insert 904 includes a ridge 910 (which may include an optional lip). However, the overall size and shape of various components in FIGS. 9A through 9D have been modified relative to FIGS. 8A through 8D.

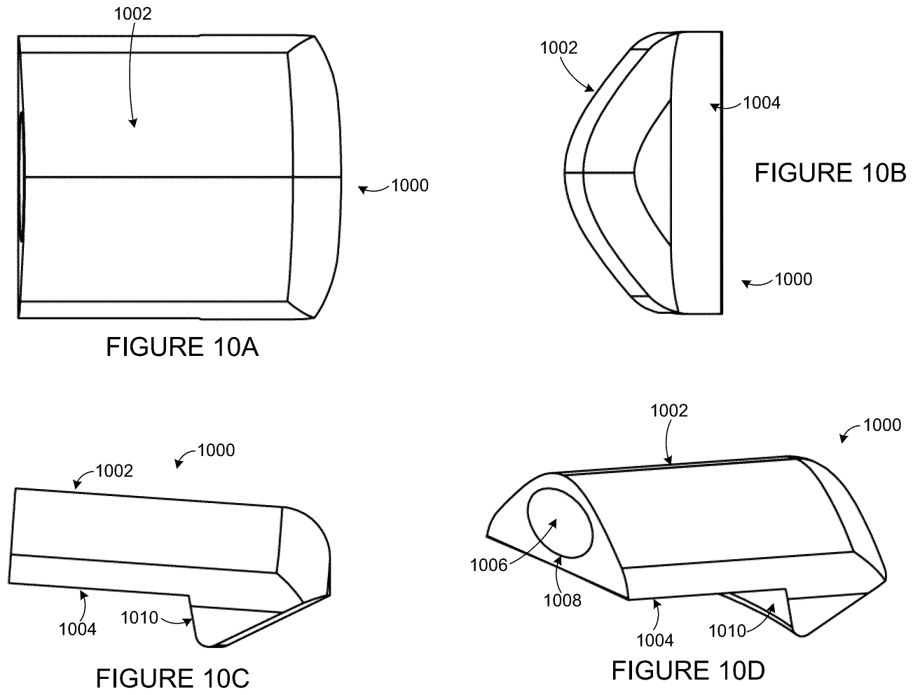

FIGS. 10A through 10D illustrate a tenth example ocular drug delivery device 1000 that is securely implantable into the anterior segment of an eye according to this disclosure. As shown in FIGS. 10A through 10D, the ocular drug delivery device 1000 is similar to the ocular drug delivery devices 800 and 900 of FIGS. 8A through 9D. For example, the ocular drug delivery device 1000 includes a reservoir 1002 and a micro-insert 1004, where the reservoir 1002 has an interior space 1006 accessible through an opening 1008. Most or all of the reservoir 1002 is positioned over the micro-insert 1004, the interior space 1006 is blocked at the other end (although this need not be the case), and the micro-insert 1004 includes a ridge 1010 (which may include an optional lip). However, the overall size and shape of various components in FIGS. 10A through 10D have been modified relative to FIGS. 8A through 9D.

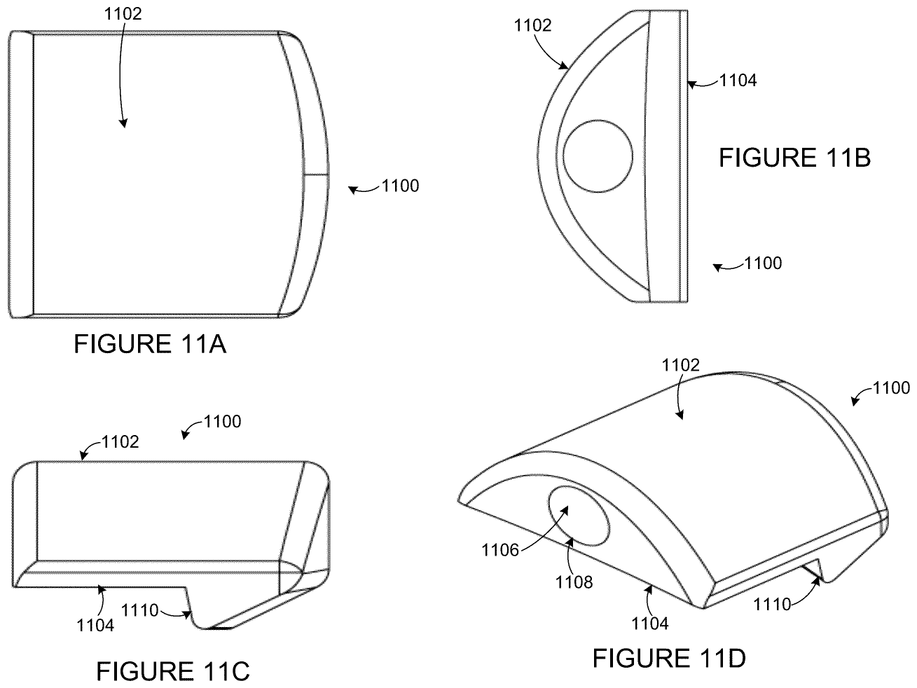

FIGS. 11A through 11D illustrate an eleventh example ocular drug delivery device 1100 that is securely implantable into the anterior segment of an eye according to this disclosure. As shown in FIGS. 11A through 11D, the ocular drug delivery device 1100 is similar to the ocular drug delivery device 1000 of FIGS. 10A through 10D. For example, the ocular drug delivery device 1100 includes a reservoir 1102 and a micro-insert 1104, where the reservoir 1102 has an interior space 1106 accessible through an opening 1108. Most or all of the reservoir 1102 is positioned over the micro-insert 1104, the interior space 1106 may be blocked at the other end (although this need not be the case), and the micro-insert 1104 includes a ridge 1110 (which may include an optional lip). However, the overall size and shape of various components in FIGS. 11A through 11D have been modified relative to FIGS. 10A through 10D.

Note that each of the ocular drug delivery devices 100-1100 described above may have any suitable size, shape, and dimensions. For example, the interior space 106-1106 of each ocular drug delivery device 100-1100 containing at least one medication could have a circular cross-sectional shape, an elliptical cross-sectional shape, a flat cross-sectional shape, or any other suitable cross-sectional shape or shapes. The interior space 106-1106 of each ocular drug delivery device 100-1100 containing the at least one medication could also follow a straight path, follow a curve path, form a disc, or have any other suitable configuration. As another example, each ocular drug delivery device 100-1100 may have a length of about 1.25 millimeters to about 1.75 millimeters, a width of about 0.9 millimeters to about 1.1 millimeters, and a height (excluding the ridge) of about 0.37 millimeters to about 0.50 millimeters. However, all of these shapes, paths, and dimensions are for illustration and explanation only and do not limit the scope of this disclosure to these specific examples.

In each ocular drug delivery device 100-1100, the reservoir 102-1102 and the micro-insert 104-1104 may each be formed from any suitable material(s). For example, the reservoir 102-1102 and the micro-insert 104-1104 may be formed using polymethyl methacrylate (“PMMA”), polyether-ether ketone (“PEEK”), or other suitable material(s). Also, each of the reservoir 102-1102 and the micro-insert 104-1104 could have any suitable size and dimensions, and ocular drug delivery devices 100-1100 of different sizes could be provided. For instance, different ocular drug delivery devices 100-1100 could have different overall sizes, reservoirs 102-1102 of different sizes, or micro-inserts 104-1104 of different sizes. Further, the reservoir 102-1102 and the micro-insert 104-1104 of each ocular drug delivery device 100-1100 may be formed separately and attached to one another (such as via an adhesive or other suitable mechanism) or formed as an integral structure. In addition, any suitable techniques may be used to form each of the ocular drug delivery devices 100-1100 or its individual parts, such as injection molding.

Although FIGS. 1A through 11D illustrate examples of ocular drug delivery devices 100-1100, various changes may be made to FIGS. 1A through 11D. For example, any suitable combination of features shown in FIGS. 1A through 11D could be used together in a single ocular drug delivery device, whether or not that specific combination of features is shown in the figures or described above. As a particular example, any of the ocular drug delivery devices 100-1100 shown in FIGS. 1A through 11D could include flat surfaces or curved surfaces, and any of the ocular drug delivery devices 100-1100 shown in FIGS. 1A through 11D could include well-defined edges or rounded edges. As other particular examples, any of the ocular drug delivery devices 100-1100 shown in FIGS. 1A through 11D may include a recess in the anterior surface of the micro-insert 104-1104 or convex and concave surfaces 310, 312 at one or more ends of the reservoir 102-1102. Also, each ocular drug delivery device 100-1100 may include any suitable number of each component shown in FIGS. 1A through 11D. Thus, for instance, an ocular drug delivery device may include multiple micro-inserts (such as two or more micro-inserts having a small angular separation) and/or multiple reservoirs (such as two or more reservoirs oriented in the same direction or in different directions). As a particular example, the number of openings 108-1108 may be based on the desired amount of medication to be delivered, such as when a larger number of openings 108-1108 can be used to provide larger amounts of at least one medication over time. In addition, each of the reservoirs 102-1102, micro-inserts 104-1104, and other components of the ocular drug delivery devices 100-1100 may have any suitable size, shape, and dimensions. For instance, as can be seen here, the shape of the reservoir in any particular ocular drug delivery device or the overall form factor of any particular ocular drug delivery device may vary widely.

It should also be noted that each ocular drug delivery device 100-1100 might be refillable or otherwise provide the ability to add new or additional medication(s) to the ocular drug delivery device 100-1100. Thus, for example, new or additional medication(s) might be injected or otherwise inserted into the reservoir of an already-implanted ocular drug delivery device 100-1100. In some cases, for instance, each ocular drug delivery device 100-1100 might have a port or other structure through which one or more medications could be injected or otherwise inserted into the reservoir. As a particular example, this might occur during a follow-up medical procedure, such as one occurring after the medical procedure in which the ocular drug delivery device 100-1100 was implanted. Depending on the circumstances, the same medication(s) as previously used could be inserted into the ocular drug delivery device 100-1100, or one or more new medication(s) could be inserted into the ocular drug delivery device 100-1100.

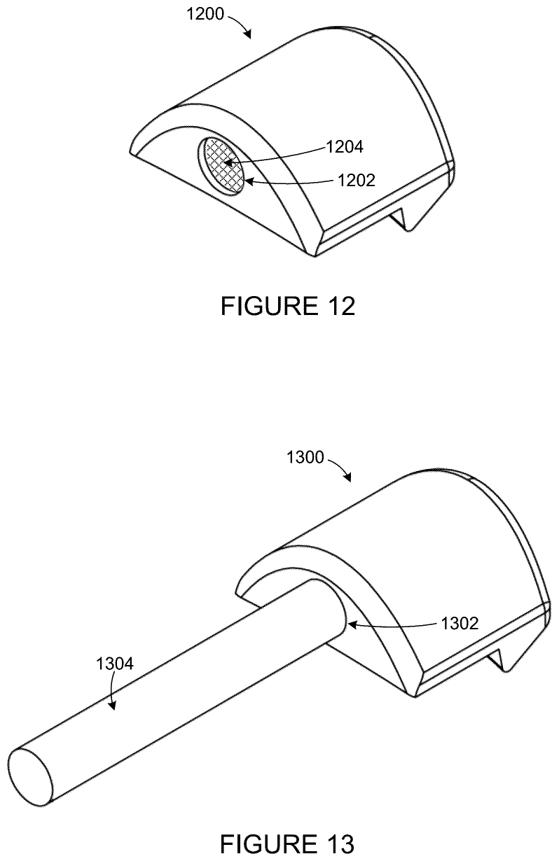

FIG. 12 illustrates an example membrane used with an ocular drug delivery device 1200 that is securely implantable into the anterior segment of an eye according to this disclosure. In this example, the ocular drug delivery device 1200 is the same as or similar to the ocular drug delivery device 1100 shown in FIGS. 11A through 11D and described above. However, the ocular drug delivery device 1200 here may be replaced with any other suitable ocular drug delivery device designed in accordance with the teachings of this disclosure, including any of the ocular drug delivery devices 100-1000 described above.

As shown in FIG. 12, the ocular drug delivery device 1200 includes at least one opening 1202, which can provide access to an interior space within a reservoir of the ocular drug delivery device 1200. The interior space can be used to hold at least one medication to be delivered to a patient's eye.

A membrane 1204 is positioned across, over, or within the opening 1202. The membrane 1204 represents a semi-permeable membrane or other membrane that allows at least one medication to be delivered into a patient's eye. The membrane 1204 may be formed from any suitable material(s) and in any suitable manner. As a particular example, the membrane 1204 may be formed using a cured polyvinyl alcohol membrane or other suitable material(s). Note that while one opening 1202 and one membrane 1204 are shown in FIG. 12, the ocular drug delivery device 1200 or other ocular drug delivery device can include multiple openings 1202 (examples of which have been described above), and each opening 1202 could have its own membrane 1204.

Although FIG. 12 illustrates one example of a membrane 1204 used with an ocular drug delivery device 1200 that is securely implantable into the anterior segment of an eye, various changes may be made to FIG. 12. For example, the size and shape of the membrane 1204 can easily vary, such as based on the size and shape of the corresponding opening 108-1108 through which the flow of at least one medication is being controlled.

FIG. 13 illustrates an example stent used with an ocular drug delivery device 1300 that is securely implantable into the anterior segment of an eye according to this disclosure. In this example, the ocular drug delivery device 1300 is the same as or similar to the ocular drug delivery device 1100 shown in FIGS. 11A through 11D and described above. However, the ocular drug delivery device 1300 here may be replaced with any other suitable ocular drug delivery device designed in accordance with the teachings of this disclosure, including any of the ocular drug delivery devices 100-1000 described above.

As shown in FIG. 13, the ocular drug delivery device 1300 includes at least one opening 1302, which can provide access to an interior space within a reservoir of the ocular drug delivery device 1300. A stent 1304 may extend from the opening 1302 (or one of the openings 1302) and away from the body of the ocular drug delivery device 1300. The stent 1304 can be said to represent an extension of the reservoir, although the stent 1304 may represent the entirety of the reservoir. The interior space of the reservoir and/or an interior space of the stent 1304 can be used to hold at least one medication to be delivered to a patient's eye. The stent 1304 can be used to provide the at least one medication to the patient's eye. Since the stent 1304 has a larger surface area (and potentially a much larger surface area) compared to the opening(s) 1302 of the ocular drug delivery device 1300, this can help to increase the amount or rate of medication delivery to the patient's eye.

In this example, the stent 1304 is relatively long, actually exceeding the depth of the remaining portions of the ocular drug delivery device 1300. However, this is not necessarily required, and the stent 1304 may extend any desired distance from the reservoir. Also, in this example, the stent 1304 is linear and extends in a straight path from the reservoir. Again, however, this is not necessarily required, and the stent 1304 may be curved, follow a segmented path, or otherwise follow a nonlinear path.

While not shown here, one or more membranes (such as one or more membranes 1204) may be positioned across, over, or within the opening 1302 and/or at one or more locations of the stent 1304. Each membrane may represent a semi-permeable membrane or other membrane that allows at least one medication to be delivered into a patient's eye. Each membrane may be formed from any suitable material(s) and in any suitable manner, such as when formed using a cured polyvinyl alcohol membrane or other suitable material(s). Also, note that while one opening 1302 and one stent 1304 are shown in FIG. 13, the ocular drug delivery device 1300 or other ocular drug delivery device can include multiple openings 1302 (examples of which have been described above), and each opening 1302 could have its own stent 1304.

It should be noted that this type of ocular drug delivery device 1300 may or may not store medication(s) within the body of the ocular drug delivery device 1300 and could potentially only store medication(s) within the stent(s) 1304. In these embodiments, the reservoir of the ocular drug delivery device 1300 could be said to reside only within the stent(s) 1304 of the ocular drug delivery device 1300. Also, it should be noted that the reservoir of the ocular drug delivery device 1300 might be replaceable or refillable. Thus, for example, new or additional medication(s) might be injected or otherwise inserted into the reservoir of an already-implanted ocular drug delivery device 1300. In some cases, for instance, the ocular drug delivery device 1300 might have a port or other structure through which one or more medications could be injected or otherwise inserted into the reservoir. As another example, one stent 1304 of an implanted ocular drug delivery device 1300 may be removed and another stent 1304 may be attached to the implanted ocular drug delivery device 1300. As a particular example, this might occur during a follow-up medical procedure, such as one occurring after the medical procedure in which the ocular drug delivery device 1300 was implanted. Depending on the circumstances, the same medication(s) as previously used could be inserted into the ocular drug delivery device 1300, or one or more new medication(s) could be inserted into the ocular drug delivery device 1300. In some embodiments, the main body of the ocular drug delivery device 1300 may be viewed as being a “docking station” for the stent 1304, such as when the stent 1304 can be positioned and inserted into the opening 1302 of the ocular drug delivery device 1300.

Although FIG. 13 illustrates one example of a stent 1304 used with an ocular drug delivery device 1300 that is securely implantable into the anterior segment of an eye, various changes may be made to FIG. 13. For example, the size and shape of the stent 1304 can easily vary.

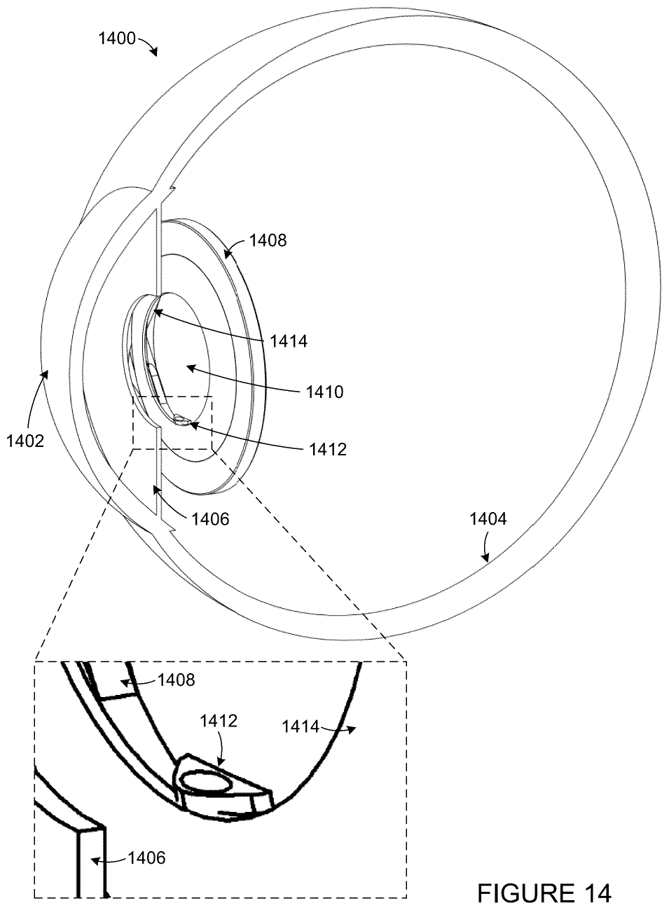

FIG. 14 illustrates an example secure implantation of an ocular drug delivery device into the anterior segment of an eye 1400 according to this disclosure. As shown in FIG. 14, the eye 1400 includes a cornea 1402, a sclera 1404, and an iris 1406. The cornea 1402 represents the clear front portion of the eye 1400 through which light passes to enter into the eye 1400. The sclera 1404 is the tough outer white portion of the eye. The iris 1406 controls the size of the eye's pupil to thereby control the amount of light from the cornea 1402 that enters into the interior of the eye 1400.

The eye 1400 also includes a capsular bag 1408, which typically holds the natural crystalline lens of the eye 1400. However, in this example, the natural crystalline lens has been removed and replaced with an intraocular lens 1410. The intraocular lens 1410 typically includes an optical lens that receives light entering the eye and focuses the light onto the retina of the eye 1400, along with micro-inserts that help to hold the intraocular lens 1410 within the capsular bag 1408 so that the optical lens of the intraocular lens 1410 is in a desired position within the eye.

An ocular drug delivery device 1412 has been placed over part of the intraocular lens 1410. As described above, the ocular drug delivery device 1412 includes a micro-insert (possibly integrated with a reservoir) that fits under an anterior leaflet 1414 of the capsular bag 1408. The anterior leaflet 1414 represents the outer portion of the front side of the capsular bag 1408 that remains after a capsulorhexis is formed in the capsular bag 1408. The positioning of the ocular drug delivery device 1412 within the eye 1400 can be seen more clearly in the enlarged portion of the eye 1400 shown in FIG. 14.

The ocular drug delivery device 1412 can represent any of the ocular drug delivery devices described above or any other suitable ocular drug delivery device designed in accordance with the teachings of this disclosure. The ocular drug delivery device 1412 is placed on the anterior surface of the intraocular lens 1410, meaning the front surface of the intraocular lens 1410 with respect to the eye 1400. The ridge of the ocular drug delivery device 1412 can contact an edge of the intraocular lens 1410, such as an outer edge of the optical lens of the intraocular lens 1410. This allows the ocular drug delivery device 1412 to be captured and confined by the anterior leaflet 1414 (and possibly attach to the anterior leaflet 1414 via fibrosis or re-fibrosis). This also allows the ridge of the ocular drug delivery device 1412 to contact the edge of the intraocular lens 1410, which can help to secure the ocular drug delivery device 1412 in place or prevent slipping of the ocular drug delivery device 1412.

Note that, in some embodiments, the surgical tool disclosed in U.S. Pat. No. 11,083,622 (which is hereby incorporated by reference in its entirety) may be used to help implant an ocular drug delivery device. For example, this tool may be used to separate part of the anterior leaflet 1414 of the patient's eye 1400 from an implanted intraocular lens 1410, thereby forming a pocket or tunnel that can receive at least part of the ocular drug delivery device 1412. As another example, this tool may be used to separate the anterior leaflet 1414 of the patient's eye 1400 from an implanted ocular drug delivery device 1412, allowing the ocular drug delivery device 1412 to be removed (and possibly replaced).

Although FIG. 14 illustrates one example of a secure implantation of an ocular drug delivery device 1412 into the anterior segment of an eye 1400, various changes may be made to FIG. 14. For example, the intraocular lens 1410 could be used with any other ocular drug delivery device. Also, there are a number of intraocular lenses available, and an ocular drug delivery device could be coupled to or used with any other suitable intraocular lens in the eye 1400.

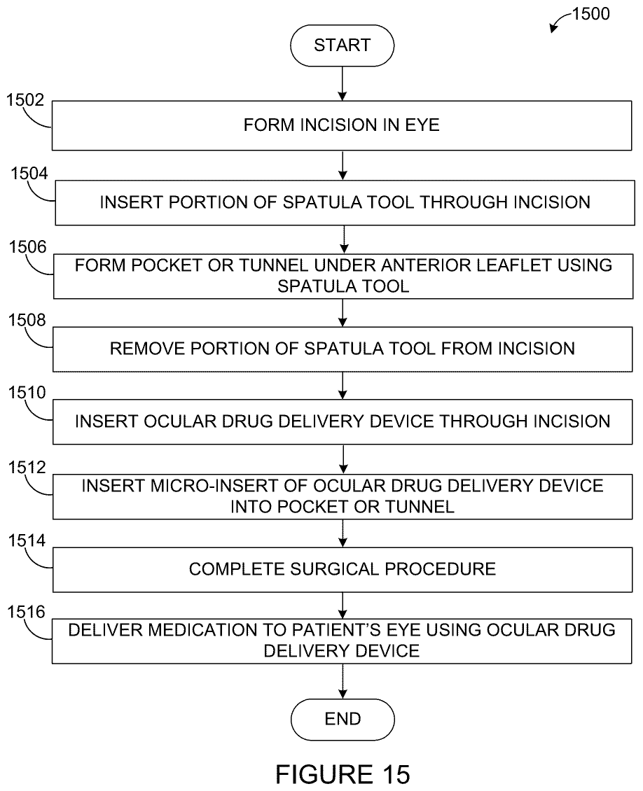

FIG. 15 illustrates an example method 1500 for using an ocular drug delivery device that is securely implantable into the anterior segment of an eye according to this disclosure. For ease of explanation, the method 1500 is described as being performed using any of the ocular drug delivery devices 100-1300 described above. However, the method 1500 may be performed using any other ocular drug delivery device designed in accordance with the teachings of this disclosure.

As shown in FIG. 15, an incision is formed in a patient's eye at step 1502. This could include, for example, a surgeon or other personnel or an automated device (such as a surgical robot) forming a small incision at the cornea-sclera junction of the patient's eye 1400. A portion of a spatula tool is inserted through the incision at step 1504, and a pocket or tunnel is formed under the anterior leaflet in the patient's eye using the spatula tool at step 1506. This could include, for example, the surgeon or other personnel or the automated device inserting the end of the spatula tool described in U.S. Pat. No. 11,083,622 through the incision in the patient's eye 1400 and using the end of the spatula tool to separate part of the anterior leaflet 1414 of the patient's eye 1400 from an intraocular lens 1410. The portion of the spatula tool is removed from the incision at step 1508.

An ocular drug delivery device is inserted through the incision in the patient's eye at step 1510. This could include, for example, the surgeon or other personnel or the automated device inserting the ocular drug delivery device into the patient's eye 1400 through the incision. The ocular drug delivery device here may be small enough to fit through the incision as-is, or the ocular drug delivery device may be rolled, folded, or otherwise reduced in cross-sectional size in order to be inserted through a smaller incision. The ocular drug delivery device here may represent any of the ocular drug delivery devices 100-1300 described above.

At least one micro-insert of the ocular drug delivery device is inserted under the anterior leaflet of the capsular wall in the patient's eye at step 1512. This could include, for example, the surgeon or other personnel or the automated device positioning the ocular drug delivery device so that its micro-insert (whether separate from or integral with its reservoir) slides under the anterior leaflet 1414 of the capsular bag 1408 into the pocket or tunnel. Note that the surgeon or other personnel or the automated device may use another tool or the spatula tool inserted through the incision to help position the ocular drug delivery device. Once inserted under the anterior leaflet 1414, the ridge of the ocular drug delivery device may be adjacent to or contact an edge of the underlying intraocular lens 1410. At that point, the surgical procedure may be completed at step 1514.

Once the surgical procedure is completed, the ocular drug delivery device can be used to deliver one or more medications to the patient's eye at step 1516. This may include, for example, the one or more medications within the reservoir of the ocular drug delivery device permeating or otherwise moving through at least one membrane of the ocular drug delivery device. Note, however, that the use of the membrane is optional, such as when other flow control mechanisms may be used or when no flow control mechanisms may be needed.

Although FIG. 15 illustrates one example of a method 1500 for using an ocular drug delivery device that is securely implantable into the anterior segment of an eye, various changes may be made to FIG. 15. For example, while shown as a series of steps, various steps in FIG. 15 could overlap, occur in parallel, occur in a different order, or occur any number of times (including zero times). Also, the use of a spatula tool to form a pocket or tunnel under the anterior leaflet is for illustration and explanation only. In general, any other suitable mechanism may be used to create space for the ocular drug delivery device under the anterior leaflet, including use of the ocular drug delivery device itself.

It may be advantageous to set forth definitions of certain words and phrases used throughout this patent document. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in this patent document should not be read as implying that any particular element, step, or function is an essential or critical element that must be included in the claim scope. Also, none of the claims is intended to invoke 35 U.S.C. § 112 (f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” “processing device,” or “controller” within a claim is understood and intended to refer to structures known to those skilled in the relevant art, as further modified or enhanced by the features of the claims themselves, and is not intended to invoke 35 U.S.C. § 112 (f).

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. An apparatus comprising:
an ocular drug delivery device comprising:
a reservoir configured to hold at least one medication for an eye; and
a micro-insert configured to be inserted under an anterior leaflet of a capsular wall in the eye;
wherein an anterior surface of the micro-insert is configured to contact an inner capsular wall surface of the anterior leaflet; and
wherein a posterior surface of the micro-insert comprises a ridge configured to contact an edge of an artificial intraocular lens.

2. The apparatus of claim 1, wherein at least part of the micro-insert extends laterally away from the reservoir.

3. The apparatus of claim 1, wherein the reservoir comprises an interior space that extends completely through the reservoir such that the reservoir has multiple openings providing access to the interior space, the interior space configured to receive the at least one medication.

4. The apparatus of claim 1, wherein the reservoir comprises an interior space that extends partially through the reservoir such that the reservoir has one opening providing access to the interior space, the interior space configured to receive the at least one medication.

5. The apparatus of claim 1, wherein the reservoir comprises an interior space configured to receive the at least one medication, the interior space extending horizontally or vertically through the reservoir.

6. The apparatus of claim 1, wherein the micro-insert comprises a recess in the anterior surface of the micro-insert, the recess extending away from the reservoir.

7. The apparatus of claim 1, wherein at least one side of the reservoir comprises a convex surface and smaller concave surfaces.

8. The apparatus of claim 1, wherein the reservoir is positioned over at least part of the micro-insert.

9. The apparatus of claim 1, wherein the reservoir is integral with the micro-insert.

10. The apparatus of claim 1, wherein the reservoir comprises:
at least one opening providing access to an interior space of the reservoir, the interior space configured to receive the at least one medication; and
at least one membrane configured to control a flow of the at least one medication through the at least one opening.

11. The apparatus of claim 1, wherein the reservoir comprises a stent configured to release the at least one medication.

12. A system comprising:
an artificial intraocular lens configured to be implanted within a capsular bag in an eye; and
an ocular drug delivery device comprising:
a reservoir configured to hold at least one medication for the eye; and
a micro-insert configured to be inserted under an anterior leaflet of a capsular wall in the eye;
wherein an anterior surface of the micro-insert is configured to contact an inner capsular wall surface of the anterior leaflet; and
wherein a posterior surface of the micro-insert comprises a ridge configured to contact an edge of the artificial intraocular lens.

13. The system of claim 12, wherein at least part of the micro-insert extends laterally away from the reservoir.

14. The system of claim 12, wherein the reservoir is positioned over at least part of the micro-insert.

15. The system of claim 12, wherein the micro-insert comprises a recess in the anterior surface of the micro-insert, the recess extending away from the reservoir.

16. The system of claim 12, wherein at least one side of the reservoir comprises a convex surface and smaller concave surfaces.

17. The system of claim 12, wherein the reservoir comprises:
at least one opening providing access to an interior space of the reservoir, the interior space configured to receive the at least one medication; and
at least one membrane configured to control a flow of the at least one medication through the at least one opening.

18. The system of claim 12, wherein the reservoir comprises a stent configured to release the at least one medication.

19. A method comprising:
forming an incision in an eye; and
inserting an ocular drug delivery device in the eye, the ocular drug delivery device comprising:
a reservoir configured to hold at least one medication for the eye; and
a micro-insert configured to be inserted under an anterior leaflet of a capsular wall in the eye;
wherein an anterior surface of the micro-insert is configured to contact an inner capsular wall surface of the anterior leaflet; and
wherein a posterior surface of the micro-insert comprises a ridge configured to contact an edge of an artificial intraocular lens.

20. The method of claim 19, further comprising:
forming a pocket or tunnel under the anterior leaflet in the eye; and
positioning at least part of the ocular drug delivery device within the pocket or tunnel.
